(19) United States
(12) Patent Application Publication
(10) Pub. No.: US 2025/0114015 A1
(43) Pub. Date: Apr. 10, 2025

(54) BIMODAL CAPACITIVE RESPIRATION SENSOR AND METHOD

(71) Applicant: Welch Allyn, Inc., Skaneateles Falls, NY (US)

(72) Inventors: Scott Martin, Camillus, NY (US); Gene J. Wolfe, Pittsford, NY (US); John A. Lane, Venice, FL (US); David E. Quinn, Auburn, NY (US)

(73) Assignee: Welch Allyn, Inc., Skaneateles Falls, NY (US)

(21) Appl. No.: 18/901,239

(22) Filed: Sep. 30, 2024

Related U.S. Application Data
(60) Provisional application No. 63/542,335, filed on Oct. 4, 2023.

(51) Int. Cl. A61B 5/091; A61B 5/00

(52) U.S. Cl. CPC A61B 5/091; A61B 5/6823; A61B 5/7264; A61B 5/7271; A61B 2560/0468; A61B 2562/0214; A61B 2562/16; A61B 2562/227

(57) ABSTRACT

A respiration monitor includes a multi-channel respiration sensor including a first electrode that is configured to measure a first capacitance signal transmitted along a first channel and a second electrode that is configured to measure a second capacitance signal transmitted along a second channel. The respiration monitor includes a processor configured to receive the first capacitance signal and the second capacitance signal, compare the first capacitance signal and the second capacitance signal to a respiration threshold, and determine which one of the first capacitance signal or the second capacitance signal is within the respiration threshold. The processor is further configured to, if one of the first capacitance signal or the second capacitance signal is outside of the respiration threshold, extrapolate only the first capacitance signal or the second capacitance signal that is within the respiration threshold to determine a tidal respiration volume.

### CROSS-REFERENCE TO RELATED APPLICATION

This application claims priority under U.S.C. § 119(e) to U.S. Provisional Application No. 63/542,335 filed on Oct. 4, 2023, entitled “BIMODAL CAPACITIVE RESPIRATION SENSOR AND METHOD,” the disclosure of which is hereby incorporated by reference in its entirety.

### FIELD OF THE DISCLOSURE

The present disclosure generally relates to a respiration sensor and, more particularly, to a respiration sensor with a multi-channel capacitive configuration that can be worn on the chest or abdomen to measure the tidal volume of a patient's respiration.

### BACKGROUND

The human respiratory system is a complex system with many variables that can affect respiration, such as clinical disease processes, changes in lung volume, air pressure, patient physical characteristics, and air flow. This complexity can make it difficult to accurately measure respiration using a single measurement technique. Clinical interpretation by direct visual observation (“clinician counting”) although common, is generally considered inferior to instrumented measurements, but instrumented measurements still have issues accurately measuring respiration in view of the above complexities.

Accordingly, the present disclosure generally relates to a respiration sensor with a multi-channel capacitive configuration that can be worn on the chest or abdomen.

### SUMMARY OF THE DISCLOSURE

According to one aspect of the present disclosure, a respiration monitor includes a multi-channel respiration sensor including a first electrode that is configured to measure a first capacitance signal transmitted along a first channel and a second electrode that is configured to measure a second capacitance signal transmitted along a second channel. The respiration monitor further includes a processor and a memory. The memory contains instructions that when executed by the processor cause the processor to receive the first capacitance signal and the second capacitance signal, compare the first capacitance signal and the second capacitance signal to a respiration threshold, and determine which one of the first capacitance signal or the second capacitance signal is within the respiration threshold. The processor is further configured to, if one of the first capacitance signal or the second capacitance signal is outside of the respiration threshold, extrapolate only the first capacitance signal or the second capacitance signal that is within the respiration threshold to determine a tidal respiration volume.

According to another aspect of the present disclosure, a respiration monitor includes a multi-channel respiration sensor including a first electrode that is configured to measure a first capacitance signal corresponding to a bending radius transmitted along a first channel and a second electrode that is configured to measure a second capacitance signal corresponding to an elongation transmitted along a second channel. The respiration monitor further includes a processor and a memory. The memory contains instructions that when executed by the processor cause the processor to receive the first capacitance signal and the second capacitance signal and extrapolate the first capacitance signal and the second capacitance signal to determine a tidal respiration volume.

According to yet another aspect of the present disclosure, a respiration monitor includes a multi-channel respiration sensor including a first electrode configured to measure a first capacitance signal corresponding to elongation in a vertical direction transmitted along a first channel and a second electrode configured to measure a second capacitance signal corresponding to an elongation in a horizontal direction transmitted along a second channel. The respiration monitor further includes a processor and a memory. The memory contains instructions that when executed by the processor cause the processor to receive the first capacitance signal and the second capacitance signal and extrapolate the first capacitance signal and the second capacitance signal to determine a tidal respiration volume.

According to still yet another aspect of the disclosure, a multi-channel respiration sensor includes a jacket defining a first passage and a second passage. A first electrode is located in the first passage and configured to measure a first capacitance signal corresponding to elongation in a vertical direction transmitted along a first channel. A second electrode is located in the second passage and configured to measure a second capacitance signal corresponding to an elongation in a horizontal direction transmitted along a second channel.

According to yet another aspect of the disclosure, a multi-channel respiration sensor includes a jacket defining a first passage and a second passage. A first electrode is located in the first passage and configured to measure a first modality along a first channel. A second electrode is located in the second passage and configured to measure a second modality along a second channel.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a front view of a respiration monitor with a multi-channel respiration sensor, according to one aspect of the present disclosure;

FIG. 2 is a front view of a multi-channel respiration sensor attached to a patient's chest, according to one aspect of the present disclosure;

FIG. 3 is a side view of a patient's chest during an inhalation process, according to one aspect of the present disclosure;

FIG. 4A is a front elevational view of a multi-channel respiration sensor of a first alternative construction, according to one aspect of the present disclosure;

FIG. 4B is a front elevational view of a multi-channel respiration sensor of a second alternative construction, according to one aspect of the present disclosure;

FIG. 4C is a front elevational view of a multi-channel respiration sensor of a third alternative construction, according to one aspect of the present disclosure;

FIG. 4D is a front elevational view of a multi-channel respiration sensor of a fourth alternative construction, according to one aspect of the present disclosure;

FIG. 4E is a front elevational view of a multi-channel respiration sensor of a fifth alternative construction, according to one aspect of the present disclosure;

FIG. 5A is a front elevational view of a display illustrating a graphical generation of a first capacitance signal and a second capacitance signal that are synchronized, according to an aspect of the present disclosure;

FIG. 5B is a front elevational view of a display illustrating a graphical generation of a first capacitance signal and a second capacitance signal that are unsynchronized, according to an aspect of the present disclosure;

FIG. 5C is a front elevational view of a display illustrating a graphical generation of a first capacitance signal within a respiration threshold and a second capacitance signal outside of a respiration threshold, according to an aspect of the present disclosure;

FIG. 6 is a schematic view of a control system for a respiration monitor, according to one aspect of the present disclosure; and

FIG. 7 is a flow chart of a method for obtaining a tidal respiration volume, according to one aspect of the present disclosure.

### DETAILED DESCRIPTION

The present illustrated embodiments reside primarily in combinations of method steps and apparatus components related to a respiration sensor with a multi-channel capacitive configuration that can be worn on the chest or abdomen to measure the tidal volume of a patient's respiration. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

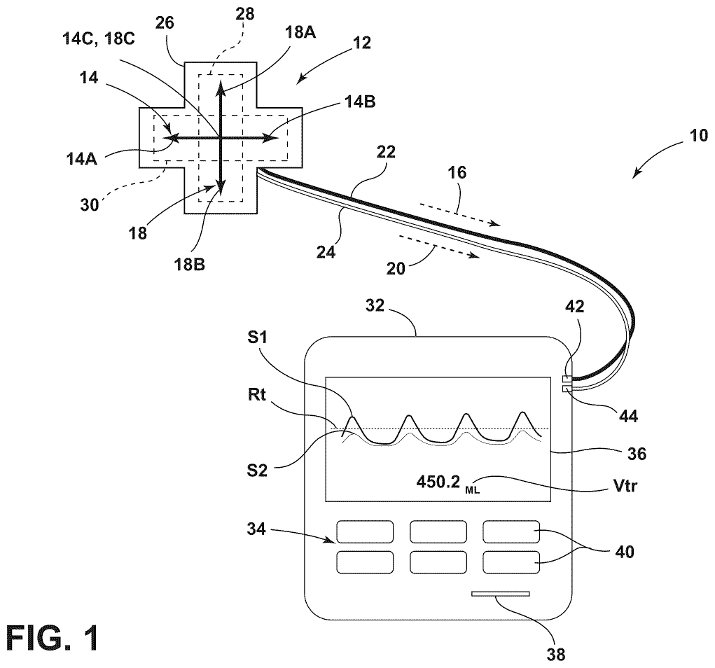

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

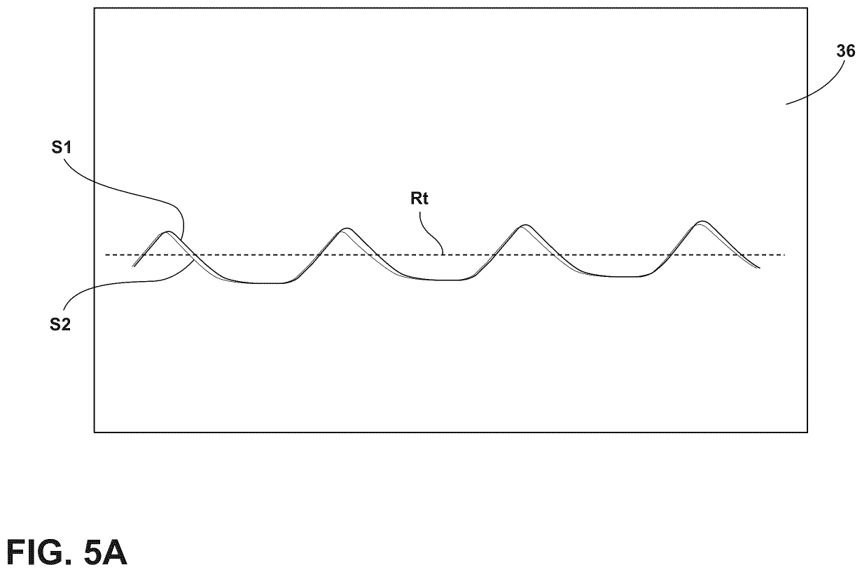

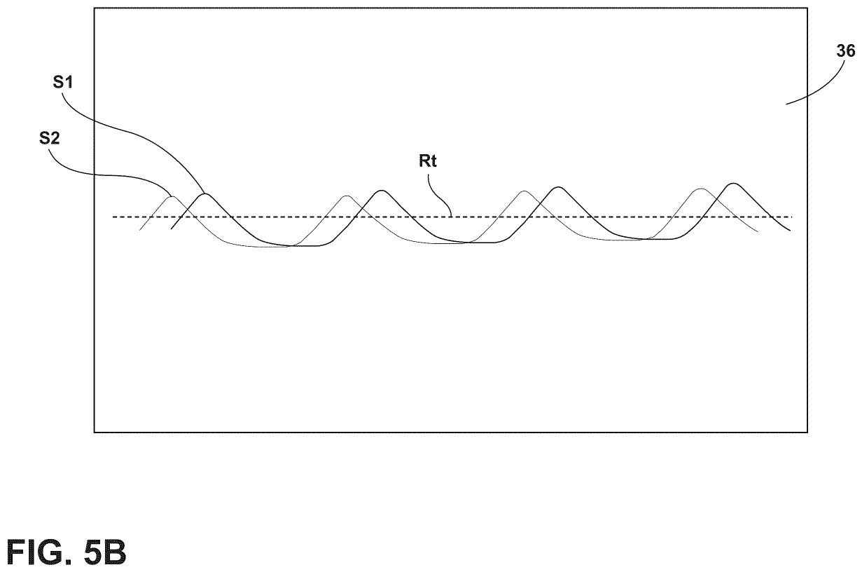

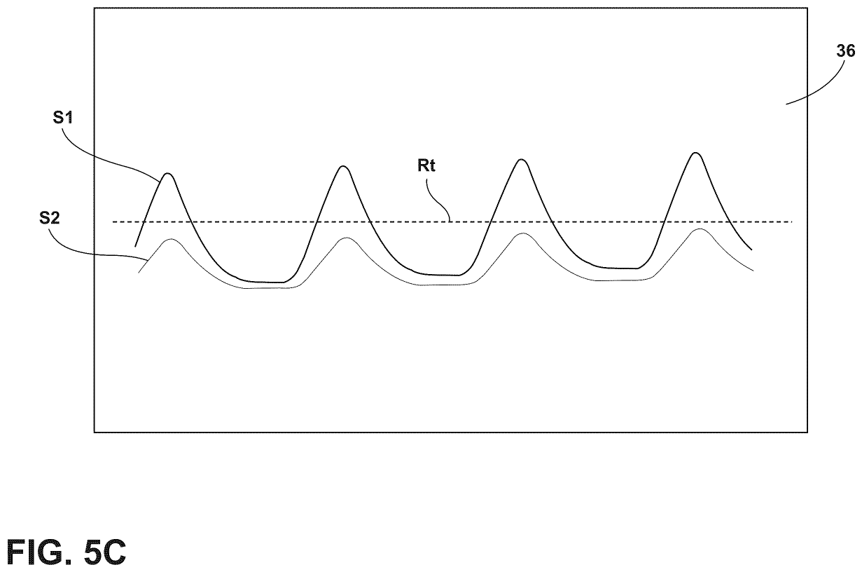

Referring to FIGS. 1-3 and 5A-7, reference number 10 generally designates a respiration monitor. The respiration monitor 10 includes a multi-channel respiration sensor 12 including a first electrode 14 that is configured to measure a first capacitance signal S1 transmitted along a first channel 16 and a second electrode 18 that is configured to measure a second capacitance signal S2 transmitted along a second channel 20. The respiration monitor 10 further includes a control system 100 (e.g., a processor 104 and a memory 106). The memory 106 contains instructions that when executed by the processor 104 cause the processor 104 to receive the first capacitance signal S1 and the second capacitance signal S1, compare the first capacitance signal S1 and the second capacitance signal S2 to a respiration threshold Rt (FIGS. 5A-5C), and determine which one of the first capacitance signal S1 or the second capacitance signal S2 is within the respiration threshold Rt. The processor 104 is further configured to not consider which of the first capacitance signal S1 or the second capacitance signal S2 is outside of the respiration threshold Rt and extrapolate only the first capacitance signal S1 or the second capacitance signal S2 that is within the respiration threshold Rt to determine a tidal respiration volume Vtr (FIG. 5C).

With reference now to FIG. 1, the first channel 16 may include a first flexible conductor 22 and the second channel 20 may include a second flexible conductor 24. The first electrode 14 and the second electrode 18 may be in a jacket 26 (e.g., a single or common jacket). The jacket 26 may be formed of textile or polymer. In some embodiments, the first electrode 14 extends from opposite first ends 14A, 14B to a first center 14C and the second electrode 18 extends from opposite second ends 18A, 18B to a second center 18C that is aligned with and overlaps the first center 14C. In this manner, the jacket 26 may define a first passage 28 and a second passage 30. In some implementations, the first passage 28 and the second passage 30 may intersect (e.g., at the first and second centers 14C, 18C) such that the first and second electrodes 14, 18 are in contact with one another. In other implementations, the first passage 28 and the second passage 30 may overlap (e.g., at the first and second centers 14C, 18C) such that the first and second electrodes 14, 18 are spaced from one another by the jacket 26. The first electrode 14 may be located in the first passage 28 and the second electrode 18 is located in the second passage 30. The first and second flexible conductors 22, 24 may extend from the first and second electrodes 14, 18 and transmit the first capacitance signal S1 and the second capacitance signal S2 to the control system 100. Alternatively, it should be appreciated that the first capacitance signal S1 and the second capacitance signal S2 may be transmitted wirelessly (e.g., without the flexible conductors 22, 24) via long-range or short-range wireless communication. In this manner, the first channel 16 may include a first wireless transmitter that transmits the first capacitance signal S1 to the control system 100 and the second channel 20 may include a second wireless transmitter that transmits the second capacitance signal S2 to the control system 100.

With continued reference to FIG. 1, the control system 100 may be located in a housing 32 that includes a user interface 34, a display 36, and a speaker 38. The user interface 34 may include a plurality of user inputs 40, such as buttons, knobs, and/or the like for controlling various operations of the control system 100, display 36, and/or speaker 38. The display 36 may generate one or more graphics, warnings, measurements, and recommendations based, for example, on the tidal respiration volume Vtr, the first capacitance signal S1, and/or the second capacitance signal S2. The speaker 38 may generate audible feedback (e.g., alerts) based, for example, on the tidal respiration volume Vtr, the first capacitance signal S1, and/or the second capacitance signal S2. More particularly, the speaker 38 may generate the audible feedback in the event of irregularities of the capacitance signals S1, S2 or the tidal respiration volume Vtr to prompt a caregiver to ensure proper connection of the electrodes 14, 18 to a patient or check for medical events (e.g., the tidal respiration volume Vtr or an aggregate of the tidal respiration volume Vtr over a period of time is abnormally low). The control system 100 may include a first receiving module 42 that receives the first capacitance signal S1 transmitted along a first channel 16 and a second receiving module 44 that receives the second capacitance signal S2 transmitted along a second channel 20 (e.g., with the flexible conductors 22, 24 or wirelessly with the transmitters). As depicted, the housing 32 and features within the housing 32 may be located in a modular hand-held unit specifically designed for the respiration monitor 10. However, in some implementations, the housing 32 may be associated with, for example, a computer, a cellular phone, the like, or another device.

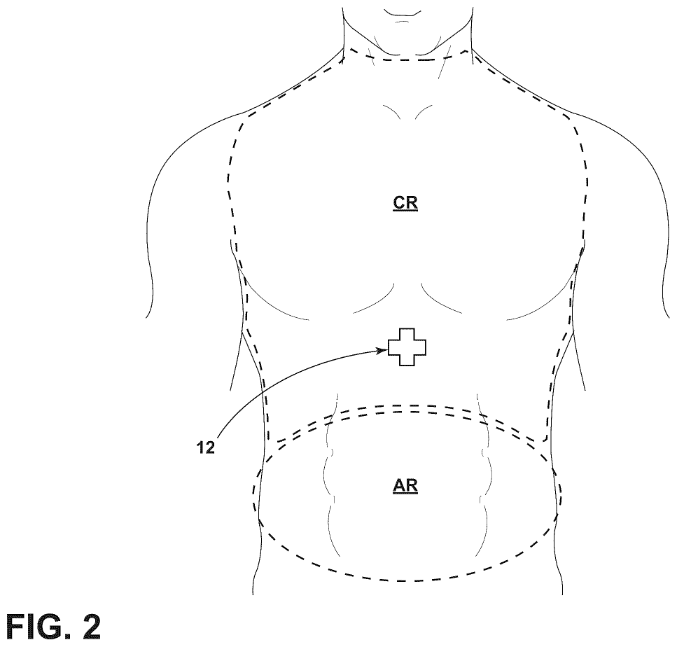

With reference now to FIG. 2, the first electrode 14 and the second electrode 18 may be configured to connect to the patient with an adhesive, tape, band, and/or the like. More particularly, in some embodiments, the jacket 26 may include an adhesive surface or be connected to a flexible band that wraps around the patient to keep the first electrode 14 and the second electrode 18 in contact with the patient. The electrodes 14, 18 and jacket 26 may be configured to be located on a chest region CR (e.g., a thorax) and/or an abdominal region AR of the patient.

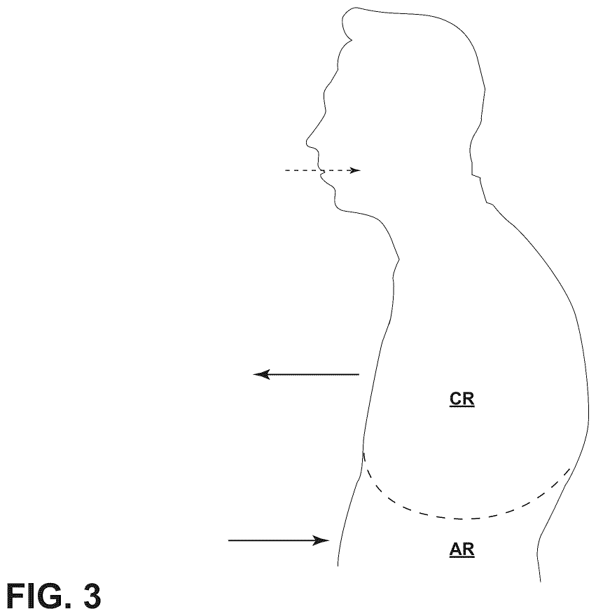

With reference now to FIGS. 2 and 3, the tidal respiration volume Vtr may be defined as a total exchanged volume occurring during an inhale and exhale. For a healthy average adult, the tidal respiration volume Vtr can be up to 7 liters and to maintain a healthy saturation of peripheral oxygen (SpO2) the tidal respiration volume Vtr is about 0.5 liters over a period of time. Several factors can affect what is considered a healthy tidal respiration volume Vtr including sex, age, weight, height, elevation, and/or other factors that affect SpO2. During respiration, the chest region CR and abdominal region AR experience relative displacement and changes in shape. For example, the chest region CR typically expands while the abdominal region AR contracts as the patient inhales. Similarly, the chest region CR typically contracts while the abdominal region AR expands as the patient exhales. Differences in relative displacement between patients can be characterized by different types of breathing, for example, shallow breathing, deep breathing, nose breathing, mouth breathing, abdominal breathing, etc. Moreover, breathing may be characterized in the expansion of the chest region CR and abdominal region AR in a vertical direction (e.g., between the feet and head) and a horizontal direction (e.g., in a direction between the shoulders).

With reference now to FIGS. 1, 2, and 4A-4E, the first and second electrodes 14, 18 may be configured to measure one or more of a plurality of modalities. For example, the first and second electrodes 14, 18 may include one or more conductive elements (e.g., plates) that exhibit different electrical characteristics, such as capacitance in response to elongation, bending (e.g. a bending radius), and/or other forms of deformation. More particularly, the first and second electrodes 14, 18 may be transverse to one another and measure elongation and/or bending in different directions relative to the chest region CR and/or abdominal region AR. In some embodiments, the first electrode 14 may extend in a first direction (e.g., between first ends 14A, 14B) and the second electrode may extend in a second direction (e.g., between second ends 18A, 18B) that is perpendicular to the first direction. In some embodiments, the first center 14C overlaps the second center 18C.

In operation, the first direction may be parallel to the vertical direction and the second direction may be parallel to the horizontal direction. The first and second electrodes 14, 18 may operate from the same modality (e.g., elongation or bend) in different directions. In other embodiments, the first and second electrodes 14, 18 may operate from different modalities (e.g., elongation and bend). For example, the first electrode 14 may operate on elongation and the second electrode 18 may operate on bend, or, alternatively, the first electrode 14 may operate on bend and the second electrode 18 may operate on elongation. When the first and second electrodes 14, 18 operate from different modalities, the first and second electrodes 14, 18 may be transverse or lying across one another at an angle (e.g., perpendicular or some other angle). In operation, the elongation of the first and/or second electrode 14, 18 may be utilized to measure expansion while the bend of the other of the first and/or second electrode 14, 18 may be utilized to measure the change in shape of the chest region CR and/or abdominal region AR. The elongation and bending radius may both be transmitted as one or both of the capacitance signals S1, S2 in the form of, for example, a unit of capacitance generation and converted into units of volume. In this manner, the tidal respiration volume Vtr can be continuously measured and monitored. It should be appreciated that one or both the first and second electrodes 14, 18 and/or other sensors may operate with different modalities other than elongation and bend, such as inductance measurements (e.g., an inductance signal), current measurements (e.g., a current signal), acoustic measurements (e.g., an acoustic signal), thoracic impedance measurement (e.g., a thoracic signal), and/or other modalities and signals. Each modality may be associated with one of the channels 16, 20 or additional channels (not shown) in embodiments with more than two modalities.

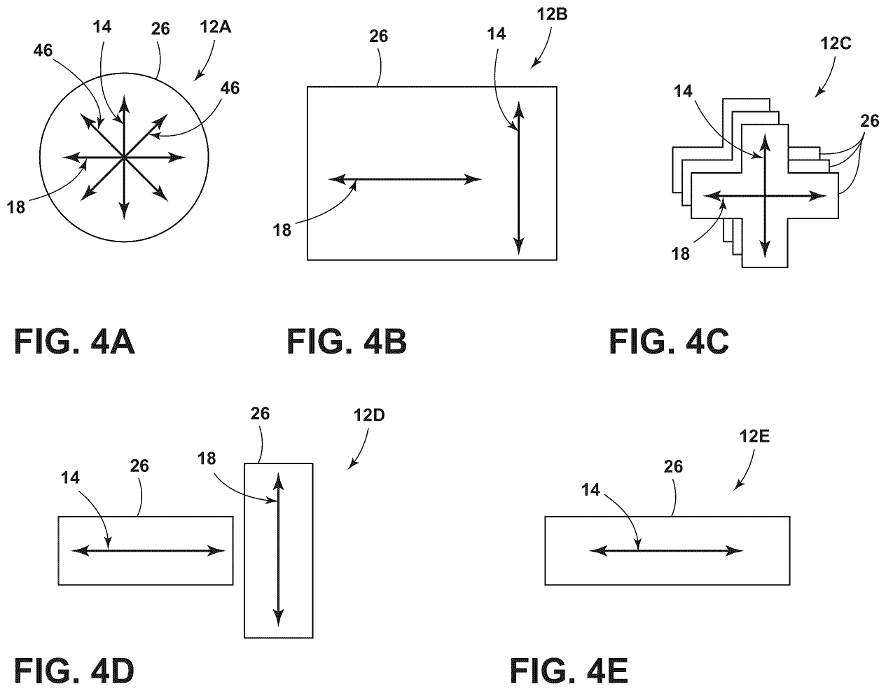

With reference now to FIGS. 4A-4E, a variety of alternative constructions of the jacket 26 and relative positioning of the first and second electrodes 14, 18 are illustrated. For example, FIG. 4A illustrates a first alternative construction of the multi-channel respiration sensor 12A. In the first alternative construction, at least one additional electrode 46 is provided in addition to the first and second electrodes 14, 18. The at least one additional electrode 46 may include a plurality of additional electrodes 46 (e.g., two or more, three or more, four or more) provided in a pattern or array. In the pattern, each electrode 14, 18, 46 may extend in different directions. For example, each electrode 14, 18, 46 may cross at the center of the other electrodes 14, 18, 46. In some implementations, each electrode 14, 18, 46 may extend in directions evenly distributed (e.g., in a circular pattern around the center of the other electrodes 14, 18, 46). The additional electrodes 46 may operate under the same (e.g., for a total of at least two modalities) or different modalities (e.g., for a total of at least three modalities) than the first and second electrodes 14, 18. Each of the additional electrodes 46 may generate different signals (e.g. third, fourth, fifth capacitance signals) that are transmitted through different channels to the control system 100. In some embodiments, the additional electrodes 46 are located in the same jacket 26. Similar to the constructions with just a first and second electrode 14, 18, the control system 100 (e.g., the processor 104) may be configured to extrapolate only whichever of the first capacitance signal, the second capacitance signal, or the one or more additional capacitance signals are within the respiration threshold to determine a tidal respiration volume. FIG. 4B illustrates a second alternative construction of the multi-channel respiration sensor 12B. In the second alternative construction, the first and second electrodes 14, 18 may extend in transverse or non-parallel directions (e.g., perpendicular) but not overlap one another and are both contained in the same jacket 26. FIG. 4C illustrates a third alternative construction of the multi-channel respiration sensor 12C. In the third alternative construction, several pairs of the first and second electrodes 14, 18 may be provided and each pair of first and second electrodes 14, 18 may be located in a different jacket 26. In this manner, the pairs of the first and second electrodes 14, 18 may be placed in different locations and/or in different orientations (e.g., electrode 14, 18 directions) within the chest region CR and/or abdominal region AR. FIG. 4D illustrates a fourth alternative construction of the multi-channel respiration sensor 12D. In the fourth alternative construction, the first and second electrodes 14, 18 may be located in different jackets 26 that may be placed in different locations and/or in different orientations (e.g., electrode 14, 18 directions) within the chest region CR and/or abdominal region AR. FIG. 4E illustrates a fifth alternative construction of the multi-channel respiration sensor 12E. In the fifth alternative construction, only one of the first or second electrodes 14, 18 may be utilized. More particularly, a single one of the first or second electrodes 14, 18 may include two or more modalities, with each modality generates the first or second capacitance signal S1, S2 (e.g., via one or more flexible conductor or wireless transmission).

With continued reference to FIGS. 4A-4E, it should be appreciated that certain features of the multi-channel respiration sensor 12 and alternative constructions 12A-12E may be incorporated into the same embodiments. For example, the first and second electrodes 14, 18 and the additional electrodes 46 may be provided in two or more jackets 26 (e.g., with each electrode 14, 18, 46 located in a different jacket 26 as illustrated in FIG. 4D or with two or more electrodes 14, 18, 46 paired in different jackets 26 as illustrated in FIG. 4C). In yet another example, the non-overlapping positioning of the first and second electrodes 14, 18 in FIG. 4B may be incorporated into the multi-channel respiration sensor 12, 12A, and 12C. In still yet another example, the single electrode 14 with two or more modalities in FIG. 4E may be incorporated with other electrodes 18, 46 that are single or multiple modalities in the multi-channel respiration sensor 12-12D.

With reference now to FIGS. 5A-5C, an example of the first and second capacitance signals S1, S2 are illustrated. In FIG. 5A, the first and second capacitance signals S1, S2 are within the respiration threshold Rt and can be utilized for extrapolating the tidal respiration volume Vtr. For example, the first and second capacitance signals S1, S2 can be averaged to improve accuracy. In FIG. 5B, the first and second capacitance signals S1, S2 are within the respiration threshold Rt but are offset. Such scenarios may be indicative of improper placement of one of the first or second electrodes 14, 18 or irregularities in breathing and may cause the generation of an alert on the display 36 or speaker 38 indicating, for example, the need to reposition the first or second electrodes 14, 18 or the patient. In FIG. 5C, only one of the first and second capacitance signals S1, S2 are within the respiration threshold Rt. In this manner, the control system 100 (e.g., the processor 104) may be configured to (e.g., if one of the signals S1, S2 are not within the respiration threshold Rt) not consider which of the first capacitance signal S1 or the second capacitance signal S2 is outside of the respiration threshold Rt and extrapolate the first capacitance signal S1 or the second capacitance signal S2 that is within the respiration threshold Rt to determine a tidal respiration volume Vtr. If one of the signals S1, S2 are not within the respiration threshold Rt, they may not be considered and either saved for predictive modeling and filtering or discarded. By incorporation of the first and second capacitance signals S1, S2, baseline shifts resulting, for example, in a patient being repositioned may be recognized by comparison with the respiration threshold Rt. In this manner, one capacitance signal S1, S2 may be utilized to offset irregularities with the other of the capacitance signal S1, S2 that is outside of (e.g., below or above) the respiration threshold Rt.

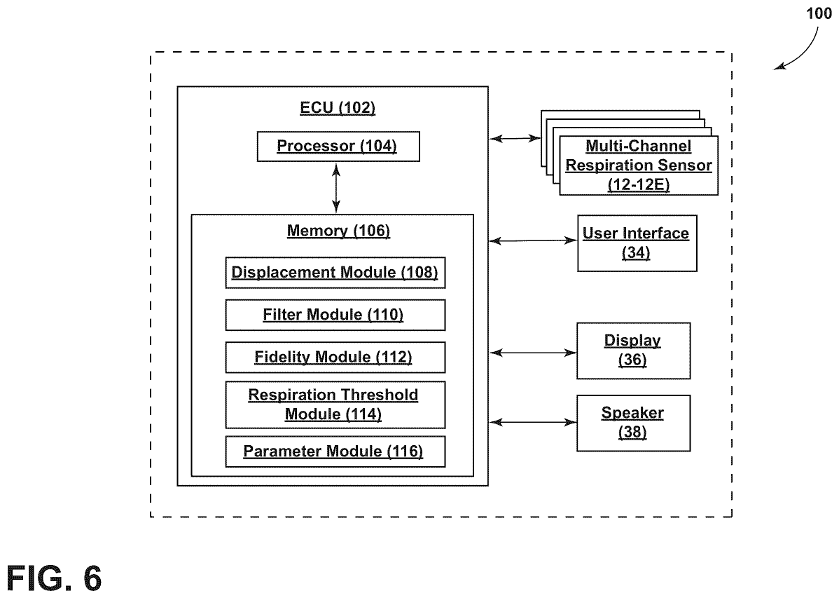

FIG. 6 schematically illustrates the control system 100. The control system 100 may include an electronic control unit (ECU) 102. The ECU 102 may include the processor 104 and the memory 106. The processor 104 may include any suitable processor 104. Additionally, or alternatively, the ECU 102 may include any suitable number of processors, in addition to or other than the processor 104. The memory 106 may comprise a single disk or a plurality of disks (e.g., hard drives) and includes a storage management module that manages one or more partitions within the memory 106. In some embodiments, memory 106 may include flash memory, semiconductor (solid-state) memory, or the like. The memory 106 may include Random Access Memory (RAM), a Read-Only Memory (ROM), Electrically Erasable Programmable Read-Only Memory (EEPROM), or a combination thereof. The memory 106 may include instructions that, when executed by the processor 104, cause the processor 104 to, at least, perform the functions associated with the components of the control system 100. The electrodes 14, 18, 46, the user interface 34, the display 36, and the speaker 38 may therefore be controlled and/or receive instructions from the ECU 102. The memory 106 may therefore include a displacement module 108, a filter module 110, a fidelity module 112, respiration threshold models 114, and a parameter module 116.

With continued reference to FIG. 6, the control system 100 is configured to receive two or more of the first and second capacitance signals S1, S2, the inductance signal, the current signal, the acoustic signal, the thoracic signal, and/or other signals that may be saved (e.g., temporarily or long term) in the displacement module 108. The two or more signals may be filtered (e.g., if they are below or above the respiration threshold Rt) by the filter module 110 to remove any noise or movement artifacts and to ensure that the signals are of sufficient quality for analysis. The filter module 110 may include techniques such as filtering (e.g., height and/or low pass), smoothing, and normalization of the signals. The fidelity module 112 may compare the signals to the respiration threshold Rt and disregard any signals that are not within the respiration threshold Rt. The respiration threshold Rt may be varied based on which of at least one respiration threshold model contained in the respiration threshold models 114 is selected. More particularly, the respiration threshold models 114 may include simulation model respiration profiles corresponding to one, two, more, or each of the patient's sex, age, weight, height, elevation, and/or other factors that affect SpO2 or tidal respiration volume Vtr that may be preselected. The parameter module 116 may include instructions to extrapolate the tidal respiration volume Vtr based on any of the signals that are filtered and within the respiration threshold Rt. Based on the tidal respiration volume Vtr the control system 100 (e.g., the processor 104) may be further configured to monitor the tidal respiration volume Vtr, visually generate a measurement of the tidal respiration volume Vtr on the display 36, and/or audibly generate a measurement of the tidal respiration volume Vtr on the speaker 38. The control system 100 (e.g., the processor 104) may be further configured to, if one of the signals is disregarded, visually generate an alert of an irregularity on the display 36, and/or audibly generate an alert of an irregularity on the speaker 38. The control system 100 (e.g., the processor 104) may be further configured still to, based on aggregating the tidal respiration volume Vtr, extrapolate an SpO2 of the patient. The control system 100 (e.g., the processor 104) may be further configured still to generate additional alerts (e.g., on the display 36 or speaker 38) if the tidal respiration volume Vtr, an aggregation of the tidal respiration volume Vtr, or the SpO2 reaches levels corresponding to unhealthy respiration. In addition, the control system 100 (e.g., the processor 104) may be further configured still to generate additional alerts in scenarios, such as FIG. 5B, where the first and second capacitance signals S1, S2 are within the respiration threshold Rt but are offset. In such scenarios, the control system 100 (e.g., the processor 104) may be configured to generate an alert on the display 36 or speaker 38 indicating, for example, the need to reposition the first or second electrodes 14, 18 or the patient before proceeding with further evaluation.

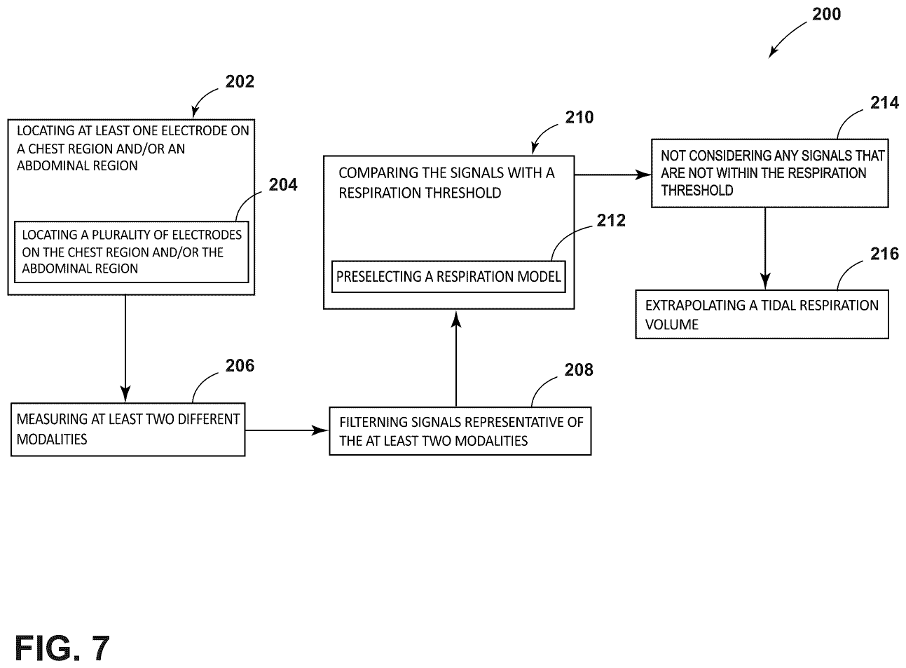

FIG. 7 illustrates a method 200 of obtaining a tidal respiration volume Vtr. At step 202, the method 200 includes locating at least one electrode 14, 18, 46 on the chest region CR and/or the abdominal region AR. Step 202 may include, at step 204, locating a plurality of electrodes 14, 18, 46 on the chest region CR and abdominal region AR or, alternatively, a single electrode 14, 18, 46 with two or more modalities on the chest region CR and abdominal region AR. At step 206, the method 200 measures at least two different modalities. In some embodiments, the modalities include one or more capacitance signals (e.g., the first and second capacitance signals S1 and S2), one or more inductance signals, one or more current signals, one or more acoustic signals, and/or one or more thoracic signals. The two different modalities may refer to a first modality that measures a first condition (e.g., bend, elongation, acoustics, or thoracic signals) and a second modality that measures a second condition that is different than the first condition. The two different modalities may refer to measuring the same conditions but in different location or in different directions. The measurements may then be transmitted as signals (e.g., to the displacement module 108).

The method 200 may further include, at step 208, filtering the signals. For example, the signals may be filtered with the filter module 110. At step 210, the method 200 may further include comparing the signals with the respiration threshold Rt. Step 210 may include, at step 212, preselecting a respiration model (e.g., via the respiration threshold models 114) based on a characteristic of the patient. The method 200 may further include at step 214 disregarding any signals that are not within the respiration threshold Rt (e.g., via the fidelity module 112). The method 200 may further include, at step 216, extrapolating the tidal respiration volume Vtr based on any signals that are not disregarded (e.g., via the parameter module 116). For example, if two or more signals are not disregarded, they may be aggregated. Step 216 may further include generating the tidal respiration volume Vtr measurement, extrapolating an SpO2 based on the tidal respiration volume Vtr over a period of time, or generating a notification (e.g., visual or auditory) to reposition the at least one electrode 14, 18, 46 or any signs of irregularities in breathing.

The disclosure herein may be further summarized in the following paragraphs and further characterized by combinations of any and all of the various aspects described therein.

According to one aspect of the present disclosure, a respiration monitor includes a multi-channel respiration sensor including a first electrode that is configured to measure a first capacitance signal transmitted along a first channel and a second electrode that is configured to measure a second capacitance signal transmitted along a second channel. The respiration monitor further includes a processor and a memory. The memory contains instructions that when executed by the processor cause the processor to receive the first capacitance signal and the second capacitance signal, compare the first capacitance signal and the second capacitance signal to a respiration threshold, and determine which one of the first capacitance signal or the second capacitance signal is within the respiration threshold. The processor is further configured to, if one of the first capacitance signal or the second capacitance signal is outside of the respiration threshold, extrapolate only the first capacitance signal or the second capacitance signal that is within the respiration threshold to determine a tidal respiration volume.

According to another aspect, the first capacitance signal corresponds to a bending radius that is transmitted along the first channel.

According to still another aspect, the second capacitance signal corresponds to an elongation that is transmitted along the second channel.

According to yet another aspect, the first capacitance signal corresponds to an elongation in a first direction that is transmitted along the first channel and the second capacitance signal corresponds to an elongation in a second direction different than the first direction that is transmitted along the second channel.

According to another aspect, the first direction is perpendicular to the second direction.

According to still another aspect, the first electrode is configured to be located along a vertical direction of a patient's chest or abdomen and the second electrode is configured to be located along a horizontal direction of the patient's chest or abdomen.

According to still yet another aspect, the first electrode and the second electrode are located in a common jacket.

According to yet another aspect, the respiration threshold is included in a model respiration profile selected from a list including at least one of a patient's sex, a patient's age, a patient's weight, or a patient's height.

According to still another aspect, the model respiration profile is selected from the list and includes at least two of the patient's sex, the patient's age, the patient's weight, or the patient's height.

According to another aspect, the respiration threshold is pre-selected from a plurality of respiration thresholds associated with a patient's sex.

According to still another aspect, the respiration threshold is pre-selected from a plurality of respiration thresholds associated with a patient's age.

According to yet another aspect, the respiration threshold is pre-selected from a plurality of respiration thresholds associated with a patient's weight.

According to another aspect, the respiration threshold is pre-selected from a plurality of respiration thresholds associated with a patient's height.

According to still another aspect, the first electrode is located in a first jacket and the second electrode is located in a second jacket.

According to yet another aspect, the respiration monitor includes at least one additional electrode with a third capacitance signal and the processor is further caused to extrapolate only whichever of the first capacitance signal, the second capacitance signal, or the third capacitance signal is within the respiration threshold to determine a tidal respiration volume.

According to another aspect of the present disclosure, a respiration monitor includes a multi-channel respiration sensor including a first electrode that is configured to measure a first capacitance signal corresponding to a bending radius transmitted along a first channel and a second electrode that is configured to measure a second capacitance signal corresponding to an elongation transmitted along a second channel. The respiration monitor further includes a processor and a memory. The memory contains instructions that when executed by the processor cause the processor to receive the first capacitance signal and the second capacitance signal and extrapolate the first capacitance signal and the second capacitance signal to determine a tidal respiration volume.

According to another aspect, the first capacitance signal corresponds to the bending radius of the first electrode in a first direction that is transmitted along the first channel and the second capacitance signal corresponds to the elongation of the second electrode in a second direction different than the first direction that is transmitted along the second channel.

According to still another aspect, the first direction is perpendicular to the second direction.

According to still yet another aspect, the first electrode and the second electrode are located in a common jacket.

According to another aspect, the jacket is formed from one of textile and polymer.

According to still another aspect, the respiration threshold is pre-selected from a plurality of respiration threshold models associated with at least one of a patient's sex, age, weight, or height.

According to yet another aspect, the first electrode defines a first center and the second electrode defines a second center that is aligned with the first center.

According to yet another aspect of the present disclosure, a respiration monitor includes a multi-channel respiration sensor including a first electrode configured to measure a first capacitance signal corresponding to elongation in a vertical direction transmitted along a first channel and a second electrode configured to measure a second capacitance signal corresponding to an elongation in a horizontal direction transmitted along a second channel. The respiration monitor further includes a processor and a memory. The memory contains instructions that when executed by the processor cause the processor to receive the first capacitance signal and the second capacitance signal and extrapolate the first capacitance signal and the second capacitance signal to determine a tidal respiration volume.

According to another aspect, the first electrode extends from opposite first ends to a first center and the second electrode extends from opposite second ends to a second center that is aligned with and overlaps the first center.

According to still another aspect, the first electrode and the second electrode are located in a common jacket.

According to still yet another aspect, the jacket is formed from one of textile and polymer.

According to another aspect, the respiration threshold is pre-selected from a plurality of respiration thresholds associated with at least one of a patient's sex, age, weight, or height.

According to still yet another aspect of the disclosure, a multi-channel respiration sensor includes a jacket defining a first passage and a second passage. A first electrode is located in the first passage and configured to measure a first capacitance signal corresponding to elongation in a vertical direction transmitted along a first channel. A second electrode is located in the second passage and configured to measure a second capacitance signal corresponding to an elongation in a horizontal direction transmitted along a second channel.

According to another aspect, the first electrode extends from opposite first ends to a first center and the second electrode extends from opposite second ends to a second center that is aligned with and overlaps the first center.

According to still another aspect, the jacket is formed from one of textile and polymer.

According to still yet another aspect, the first channel includes a first flexible conductor extending from the jacket and second channel includes a second flexible conductor extending from the jacket.

According to yet another aspect of the disclosure, a multi-channel respiration sensor includes a jacket defining a first passage and a second passage. A first electrode is located in the first passage and configured to measure a first modality along a first channel. A second electrode is located in the second passage and configured to measure a second modality along a second channel.

According to another aspect, the first electrode extends in a first direction and the second electrode extends in a second direction that is perpendicular to the first direction.

According to another aspect, the first modality corresponds to elongation of the first electrode the first direction and the second modality corresponds to elongation of the second electrode in the second direction.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A respiration monitor comprising:
a multi-channel respiration sensor including a first electrode configured to measure a first capacitance signal that is transmitted along a first channel and a second electrode configured to measure a second capacitance signal that is transmitted along a second channel; and
a processor and a memory, the memory containing instructions that when executed by the processor cause the processor to:
receive the first capacitance signal and the second capacitance signal;
compare the first capacitance signal and the second capacitance signal to a respiration threshold;
determine which one of the first capacitance signal or the second capacitance signal is within the respiration threshold; and
if one of the first capacitance signal or the second capacitance signal is outside of the respiration threshold, extrapolate only the first capacitance signal or the second capacitance signal that is within the respiration threshold to determine a tidal respiration volume.

2. The respiration monitor of claim 1, wherein the first capacitance signal corresponds to a bending radius that is transmitted along the first channel.

3. The respiration monitor of claim 2, wherein the second capacitance signal corresponds to an elongation that is transmitted along the second channel.

4. The respiration monitor of claim 1, wherein the first capacitance signal corresponds to an elongation of the first electrode in a first direction that is transmitted along the first channel and the second capacitance signal corresponds to an elongation of the second electrode in a second direction different than the first direction that is transmitted along the second channel.

5. The respiration monitor of claim 4, wherein the first direction is perpendicular to the second direction.

6. The respiration monitor of claim 5, wherein the first electrode is configured to be located along a vertical direction of a patient's chest or abdomen and the second electrode is configured to be located along a horizontal direction of the patient's chest or abdomen.

7. The respiration monitor of claim 6, wherein the respiration threshold is included in a model respiration profile selected from a list including at least one of a patient's sex, a patient's age, a patient's weight, or a patient's height.

8. The respiration monitor of claim 7, wherein the model respiration profile is selected from the list and includes at least two of the patient's sex, the patient's age, the patient's weight, or the patient's height.

9. The respiration monitor of claim 1, wherein the first electrode and the second electrode are located in a common jacket.

10. The respiration monitor of claim 1, wherein the first electrode is located in a first jacket and the second electrode is located in a second jacket.

11. The respiration monitor of claim 1, further including at least one additional electrode with a third capacitance signal and the processor is further caused to:
extrapolate only whichever of the first capacitance signal, the second capacitance signal, or the third capacitance signal are within the respiration threshold to determine a tidal respiration volume.

12. A respiration monitor comprising:
a multi-channel respiration sensor including a first electrode configured to measure a first capacitance signal corresponding to a bending radius that is transmitted along a first channel and a second electrode configured to measure a second capacitance signal corresponding to an elongation that is transmitted along a second channel; and
a processor and a memory, the memory containing instructions that when executed by the processor cause the processor to:
receive the first capacitance signal and the second capacitance signal; and
extrapolate the first capacitance signal and the second capacitance signal to determine a tidal respiration volume.

13. The respiration monitor of claim 12, wherein the first capacitance signal corresponds to the bending radius of the first electrode in a first direction that is transmitted along the first channel and the second capacitance signal corresponds to the elongation of the second electrode in a second direction different than the first direction that is transmitted along the second channel.

14. The respiration monitor of claim 13, wherein the first direction is perpendicular to the second direction.

15. The respiration monitor of claim 12, wherein the first electrode and the second electrode are located in a common jacket.

16. The respiration monitor of claim 15, wherein the first electrode defines a first center and the second electrode defines a second center that is aligned with the first center.

17. The respiration monitor of claim 15, wherein the jacket is formed from one of textile and polymer.

18. A multi-channel respiration sensor comprising:
a jacket defining a first passage and a second passage;
a first electrode located in the first passage to measure a first capacitance signal corresponding to a first modality that is transmitted along a first channel; and
a second electrode located in the second passage to measure a second capacitance signal corresponding a second modality that is transmitted along a second channel.

19. The respiration monitor of claim 18, wherein the first electrode extends in a first direction and the second electrode extends in a second direction that is perpendicular to the first direction.

20. The respiration monitor of claim 19, wherein the first modality corresponds to elongation of the first electrode the first direction and the second modality corresponds to elongation of the second electrode in the second direction.
